(19) United States
(12) Patent Application Publication
(10) Pub. No.: US 2019/0008907 A1
(43) Pub. Date: Jan. 10, 2019

(54) COMPOSITIONS FOR MANAGEMENT OF WOUNDS, SKIN DISEASES, DEHYDRATION, CHRONIC DISEASES, AND RESPIRATORY DISEASES

(71) Applicant: Noori AL-WAILI, Richmond Hill, NY (US)

(72) Inventor: Noori AL-WAILI, Richmond Hill, NY (US)

(21) Appl. No.: 16/023,050

(22) Filed: Jun. 29, 2018

Related U.S. Application Data
(60) Provisional application No. 62/529,615, filed on Jul. 7, 2017.

(51) Int. Cl. A61K 35/644; A61K 36/9066; A61K 9/00; A61K 31/122; A61K 36/71; A61P 31/04

(52) U.S. Cl. CPC A61K 35/644; A61K 36/9066; A61K 9/0014; A61K 9/0029; A61P 31/04; A61K 9/0053; A61K 31/122; A61K 36/71; A61K 9/007

(57) ABSTRACT

A composition including a mixture of honey, curcumin, propolis or CAPE or any two of them is used to treat a variety of medical conditions. The composition can be administered topically, orally, by inhalation or parenterally. The composition is useful in treating wounds, infections, diabetes, skin conditions, renal conditions, dehydration, malnutrition, hypertension and various other conditions. The mixture of honey, curcumin, and propolis or CAPE, either alone or in combination with other natural products is more effective than the administration of single ingredient.

### CROSS-REFERENCE TO RELATED APPLICATIONS

This application claims priority under 35 USC 119(e) of U.S. Provisional Application No. 62/529,615, filed on Jul. 7, 2017, the disclosure of which is herein incorporated by reference.

### BACKGROUND OF THE INVENTION

### 1. Field of the Invention

The present invention relates to compositions for management of wounds, skin diseases, dehydration, chronic diseases, and respiratory diseases. In particular, the invention relates to using mixture of honey, propolis and curcumin either alone or in combination with other products for the treatment of various afflictions of the human and animal body.

### 2. The Prior Art

It is well known that various bee products and curcumin have healing properties and are suitable for treating wounds, skin diseases and other afflictions.

Honey has served as a common base for wound healing compositions since ancient times. Honey has been found to have antibacterial, antifungal, antiviral, anti-inflammatory, antihypertensive, antioxidant, antitumor, cardioprotective, hepatoprotective, and hypoglycemic properties.

Curcumin belongs to the family of natural compounds collectively called curcuminoids and it possesses remarkable beneficial anti-oxidant, anti-inflammatory, anti-microbial, anti-cancer, and neuroprotective properties. It is the most active component of rhizome of Curcuma longa L. (common name: turmeric). Furthermore, curcumin is effective in the treatment of chronic pain, inflammatory dermatoses, acceleration of wound closure, skin infections, as well as dyspigmentation. Curcumin has beneficial impact in the wound healing process. It stimulated the production of the growth factors involved in the wound healing process. curcumin could protect tissues from oxidative damage and significantly improve tissue remodeling. It decreases pain and inflammation in arthritis and improves insulin sensitivity in diabetes. Curcumin improved rates of inflammatory cells, collagen deposition, angiogenesis, granulation tissue formation and epithelialization

Curcumin has low aqueous solubility, poor tissue absorption, rapid metabolism and short plasma half-life. However, various topical formulations of curcumin such as films, fibers, emulsion, hydrogels and different nanoformulations have been developed for targeted delivery of curcumin at wounded sites. For example, a novel dual drug co-loaded in situ gel-forming nanoparticle/hydrogel system which acted as a sustained drug depot for curcumin. Curcumin conjugated to hyaluronic acid, which is a natural polysaccharide known to influence the healing process. Furthermore, a water-soluble liposomal curcumin system was developed to increase its bioavailability after intravenous administration.

Propolis is a natural plant product collected by Apis mellifera honeybees from plants. It is a resinous mixture of botanical balsams and resin with digestive enzymes of bees. It contains more than 300 natural compounds including polyphenols, phenolic aldehydes, amino acids, steroids, sequiterpene-quinones, coumarins, and inorganic compounds. Propolis consists of approximately 55% resinous and balm substances, approximately 30% wax, 5 to 10% essential oils, 2 to 5% pollen, vitamins and microelements. Propolis is relatively non-toxic, with a no-effect level in a dose of 1400 mg/kg body weight/day (Burdock G., Review of the biological properties and toxicity of bee propolis (propolis). Food Chem Toxicol 1998; 36: 347-363).

Propolis has antioxidant, immune-modulatory effects, antimicrobial, anti-inflammatory, antitumor, antiulcer, hepato and cardio-protective properties. Propolis also has antimicrobial activity and has renal and hepatic protection property.

CAPE (caffeic acid phenethyl ester) is an active phenolic part of propolis and has antitumoral, antiproliferative, anti-inflammatory, antineoplastic, and antioxidant properties. The molecular weight and empirical formula of CAPE are 284.3 g/mol and C₁₇H₁₆O₄. All flavonoids within propolis are reported to have a low order of acute oral toxicity with a reported LD50 of 8-40 g/kg. Similarly, a safe dose in humans is estimated as 1.4 mg/kg body weight/day, or approximately 70 mg/day (Burdock G., Review of the biological properties and toxicity of bee propolis (propolis). Food Chem Toxicol 1998; 36: 347-363).

Olive oil contains 95 to 99% acyl glyceroles, 0.5 to 1.5% unsaponifyable substances and 0.1 to 3% free fatty acids, glycerides, secciridoids and flavonoids. The main fatty acids are oleic acid, palmitic acid and linoleic acid. Furthermore, it contains phenolic compounds, carbohydrates, steroles, triterpene alcohols, hydroxy triterpenic acids, tocopheroles, phospholipids, carotinoids, chlorophyll and pheophytines.

Garlic (Allium sativum L.) is one of the world's oldest medicines and it has biological activities, including anti-carcinogenic, antiatherosclerotic, antithrombotic, antimicrobial, antiinflammatory and antioxidant effects.

Nigella sativa belongs to the botanical family of Ranunculaceae and commonly grows in the Eastern Europe, Middle East, and Western Asia. It is a small shrub with tapering green leaves and rosaceous white and purplish flowers. Its ripe fruit contains tiny seeds, dark black in color. Nigella sativa is native to Southern Europe, North Africa and Southwest Asia, Middle Eastern Mediterranean region, India, Pakistan, Syria, and Turkey. Among Muslims, it is considered as one of the greatest forms of healing medicine available as was mentioned that black seed is the remedy for all diseases except death in one of the Prophetic hadith. It is also recommended for use on regular basis in Tibb-e-Nabwi (Prophetic Medicine). Nigella sativa seeds contain an essential oil (0.4-2.5%), fixed oil (36-48%), alkaloids, saponin and proteins. Nigella sativa oil was shown to contain thymoquinone which is the main bioactive component (27.8-57.0%) of the essential oil of the black seed. Toxicity studies on laboratory animals have reported that nigella sativa oil and thymoquinone are quite safe, mainly when given orally.

Data has showed that nigella sativa has diuretic, antihypertensive, antidiabetic, anticancer and immunomodulatory, analgesic, antimicrobial, anthelmintics, analgesics and anti-inflammatory, spasmolytic, bronchodilator, gastroprotective, hepatoprotective, renal protective and antioxidant properties. The seeds of nigella sativa are widely used in the treatment of various diseases like bronchitis, asthma, diarrhea, rheumatism and skin disorders. It is also used as liver tonic, digestive, anti-diarrheal, appetite stimulant, to increase milk production in nursing mothers to fight parasitic infections, and to support immune system. Nigella sativa oil and thymoquinone have been used as anti-inflammatory, antioxidant and anticancer therapeutic Agents.

Bee venom therapy is thousands of years old and it involves the application of live bee stings to the skin or the injection of bee venom into the skin. Bee venom is produced by honeybees (Apis mellifera). Bee venom has anti-inflammatory, antibacterial, and anti-rheumatic activities. It has been used in arthritis, rheumatism, back pain, multiple sclerosis, cancer, and skin diseases. It relieves pain and inflammation and it has an immune response enhancing effect. It contains melittin, apamin, adolapin, phospholipase A2, mast cell degranulating peptide, α-D-glucosidase, biologically activity amines, hyaluronidase, acid phosphomonesterase, and lysophospholipase. Melittin has anti-inflammatory and antioxidant activity.

Syzygium aromaticum (synonym: Eugenia cariophylata) is commonly known as clove. It is a medium sized tree (8-12 m) native from the Maluku islands in east Indonesia. Cloves are the aromatic flower buds of a tree in the family Myrtaceae. Clove represents one of the major vegetal sources of phenolic compounds as flavonoids, hidroxibenzoic acids, hidroxicinamic acids and hidroxiphenyl propens. Eugenol is the main bioactive compound of clove. Other phenolic acids found in clove are the caffeic, ferulic, elagic and salicylic acids. Flavonoids as kaempferol, quercetin and its derivates (glycosilated) are also found in clove in lower concentrations. Concentrations up to 18% of essential oil can be found in the clove flower buds. It was estimated that 89% of the clove essential oil is eugenol and 5% to 15% is eugenol acetate and β-cariofileno.

The essential oil extracted from flower buds of clove is used as a topical application to relieve pain and to promote healing. Its main constituents are phenylpropanoids such as carvacrol, thymol, eugenol and cinnamaldehyde. In addition to its antimicrobial, antioxidant, antifungal and antiviral activity (herpes simplex), clove essential oil possesses anti-inflammatory, cytotoxic, insect repellent and anesthetic properties. Syzygium aromaticum (clove) has an anti-microbial activity.

Acacia Arabica gum is a traditional oral hygiene substance that has been used for centuries by many communities in the Middle East and North Africa. It consists mainly of Arabica, a complex mixture of the calcium, magnesium and potassium salts of Arabic acid. There are also other constituents such as tannins, cyanogenic glycosides, oxidases, peroxidases and pectinases; all of which have been shown individually to exhibit antimicrobial properties.

Gum Arabic is mixture of polysaccharides, oligosaccharides and glycoproteins. It is exudates of Acacia Senegal/seyal trees. It is water soluble; therefore, it is used as an emulsifier, thickening substance and flavor stabilizer in many pharmaceutical and food industries.

Bees collect pollen from plant anthers, mix it with a small dose of the secretion from salivary glands or nectar, and place it in corbiculae which are situated on the tibia of their hind legs. The chemical composition of bee pollen depends strongly on the plant source and geographic origin, together with other factors such as climatic conditions, soil type, and bees' race and activities. Bee pollen contains about 250 substances including amino acids, lipids (triglycerides, phospholipids), protein, vitamins, macro- and micronutrients, phenolic compounds, enzymes, and coenzymes and flavonoids.

Bee pollen contains 22.7% of protein on average (10.4% of essential amino acids, significant amounts of nucleic acids), digestible carbohydrates (30.8% on average), essential fatty acids, phenolic compounds (flavonoids, leukotrienes, catechins, and phenolic acids), multivitamins 0.7%, calcium, phosphorus, magnesium, sodium, and potassium, iron, copper, zinc, manganese, silicon, and selenium 1.6%. Bee pollen has antifungal, antimicrobial, antiviral, anti-inflammatory, immune-stimulating, and local analgesic and also facilitates the granulation process of the burn wound healing.

Royal jelly is a mixture of yellow-white creamy and acidic secretions produced by the worker honeybees using their mandibular and hypopharingeal glands to supply food for a queen honeybee. It is rich in protein, amino acids, fatty acids, calcium, iron, minerals, vitamins, and carbohydrates. 10-HAD is the most active ingredient. Royal jelly has antimicrobial properties against yeast, Gram-negative and Gram-positive bacteria. Royal jelly stimulates immune-co-potent cell proliferation and production of antibodies in mice.

Flaxseed (linseed, Linum usitatissimum), is the richest plant source of α-linolenic acid (50%-62% of flaxseed oil, or 22% of whole flaxseed) and lignans (a class of phytoestrogen, range: 0.2-13.3 mg/g flaxseed) and it contains dietary fiber (28% by weight), a third of which is soluble fiber. Whole flaxseed or flaxseed oil are included in the general oral composition and renal healing composition.

Juglans regia Linn. (Juglandaceae) is a valuable medicinal plant and various parts of the plant have been used for management of diseases in different cultures. Walnuts contain unsaturated fatty acids, proteins, fiber, vitamins, minerals, phytosterols and polyphenols. Furthermore, walnuts contain a number of potentially neuroprotective compounds like vitamin E, folate, melatonin, several antioxidative polyphenols and significant amounts of ω-3 fatty acids. Its health benefits include reduced risk of cardiovascular disease, certain cancer and type II diabetes, and amelioration of symptoms of neurological disorders. It is rich in polyphenols and other phytochemicals, polyunsaturated and fatty acids with a particularly high ω3: ω6 ratio—the highest among all the tree nuts.

While each of these natural ingredients alone have healing properties, it would be desirable to produce a product that increases the effectiveness of the natural ingredients beyond what has already been documented.

### SUMMARY OF THE INVENTION

It is an object of the invention to provide a healing substance that exhibits superior healing properties as compared to the natural products alone described above.

This object is accomplished by a product that contains a mixture of different types of natural products. By mixing different natural substances, the various beneficial properties of the different substances are combined and enhance the healing process.

The honey can be honey of different color such as dark honey light honey, yellow honey or white honey, or with any other color; of different floral origin either unifloral or multiflora, and can be the same honey made by different bee species.

The formulations can include total honey or honey extract, such as water, alcohol or ethyl acetate extracts. The formulations can include crude propolis, or propolis extract. The formulations can include total curcumin powder or curcumin extract. Curcumin could be used in different formulations such as films, fibers, emulsion, hydrogels and different nanoformulations, conjugated to hyaluronic acid, and a water-soluble liposomal curcumin system to increase its bioavailability after skin, oral and systemic administration such as intravenous.

The honey-propolis-curcumin formulations are useful for the prevention/treatment of, inter alia, wounds, ulcers, burns, skin infection, and related skin disorders such as contact dermatitis, eczema, psoriasis, wrinkle and other skin diseases (inflammatory, infectious or allergic). The compositions are also antimicrobial to fungus, mold, bacteria, viruses and yeasts, indicating potential to treat skin, wound and ulcer infections in human or animals.

The invention also comprises oral compositions for treatment of, inter alia, anemia, enhancement of immunity, potentiating the effects of antibiotics and chemotherapy, for management of dyslipidemia, diabetes mellitus, cardiovascular diseases, H. pylori gastritis or ulcers, dyspepsia, acute and chronic kidney diseases and hypertension, and for prevention or amelioration of toxic effects of heavy metals or chemotherapies or radio therapy.

The invention also comprises intravenous compositions for protection of, inter alia, liver, kidney, bone marrow when used with chemotherapy, cytotoxic drugs, and immunosuppressive agents, or used in inflammatory diseases, infectious diseases or sepsis or dehydration or malnutrition or part of cancer management.

The invention also comprises inhalation compositions for the management of infectious respiratory diseases, sinusitis, and lung obstructive diseases or malignancies.

Although honey, propolis, and curcumin alone or mixed with other substances are already known in the medical science, yet so far mixtures of these substances has never been used in topical, parenteral, inhalational or oral treatment.

The objective of the present invention is to provide a natural agent for nutritional and medicinal use, which has a broad application in human and animals, which also composed of multiple natural ingredients and, therefore, is lack of adverse effects, without contraindication, and well tolerated.

A further objective of the present invention is to use the natural ingredients in doses, which are suitable for human or animal use and without side effect.

A particular advantageous use of the mixture of honey, propolis and curcumin (or any two of them) alone or in combination with one or more than one of the following natural substances such as bee pollen, royal jelly, nigella sativa, thymoquinone, bee venom or melittin, garlic or aged garlic or aged garlic extract (AGE), flaxseed or flaxseed oil, green tea, fenugreek seeds, walnuts, ginger, acacia Arabica, clove or olive oil will provide a composition with high potency, wide spectrum of medicinal and nutritional use, low cost, lack of adverse effects, well tolerability, ability to potentiate the use of chemotherapy, antibiotics and other modern interventions, improving circulation, and ameliorating side effects of immunosuppressive and chemotherapeutic agents as well as radiotherapy.

A further advantageous use of the substance mixture according to the invention is that treatment with these substances includes a decrease in length of hospital stay, decrease in complications of chronic diseases such as diabetic nephropathy, diabetic retinopathy, hypertensive heart disease, burn scars and deformities, wound scars, and decreasing further invasive interventions.

A further advantageous use of the substance mixture according to the invention consists in preparing compositions for, not limited to, skin care, skin diseases, diabetes mellitus, hypertension, cardiovascular diseases, kidney diseases, urinary calculus, infections (bacterial, viral and parasitic), malignancies, malnutrition, and respiratory diseases.

A further advantageous use of the substance mixture according to the invention consists in preparing compositions for to be used in topical, parenteral, inhalational or oral treatment.

Another advantageous use of the substance mixture according to the invention includes providing biological systems with potent antioxidant capacity, potent antimicrobial intervention, potent anti-inflammatory agents, potent analgesic intervention, and potent nutritional support more than using individual ingredients. There is no intervention in modern medicine available with all these properties together.

Further, it is an object of the present invention to provide a composition with a base of honey, propolis and curcumin which is suitable for the treatment of parasitic diseases such as Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. The formulations can include total honey or honey extract, such as water, alcohol or ethyl acetate extracts.

The different compositions can be administered topically, orally, parenterally, by inhalation (nasal or oral), vaginally, or rectally in dosage unit formulations containing conventional nontoxic pharmaceutically acceptable carriers, adjuvants, and vehicles, as desired. The term parenteral as used herein includes intravenous, intramuscular, intraperitoneal, intrasternal injection, or infusion techniques.

The wound/skin healer composition is a composition for topical application in or on a wound or ulcer that is believed to promote healing and tissue growth, provide nutritional factors and antioxidants, and to eradicate infection and accelerate wound healing. The composition includes mixture of honey, propolis and curcumin (or combination any two of them) either alone or combined with bee venom or melittin, bee pollen or mixed bee pollen, royal jelly or mixed royal jelly, acacia Arabic, olive oil, Nigella Sativa or thymoquinone, clove, and garlic or aged garlic or AGE. Acacia Arabica is used in heavy exudate, bleeding wounds, and also to increase viscosity of the formulae. It has antioxidant and antimicrobial effects. Multiple compositions can be prepared with mixing any two or more ingredients including honey, propolis and curcumin to achieve the same goals.

The powders of crude ingredients or their extracts are mixed into the viscous honey base, and the composition may be applied directly to the wound or ulcer or skin diseases, or the composition may be applied to sterile gauze or incorporated in any wound materials such as alginate, foams, hydrocolloids, interactive dressing, or composite dressing and the wound or ulcer, particularly an open surgical wound or deep ulcer, may be packed with the gauze.

The amount of the wound/skin compositions to be administered and the frequency of administration and period of the treatment depend on the type, location, chronicity, and severity of the wound, ulcer or infection. The compositions may be presented in unit-dose or multi-dose containers. And they may include additional ingredients that are conventional in the art with regard to topical formulations. The healing herein includes curing, alleviating, reversing, palliative and prophylactic treatment of the condition.

The main advantage of the honey, propolis and curcumin composition according to the invention includes:
1—Natural products and available everywhere,
2—Safe and almost no side effects,
3—Synergistic effects as a wound healer as well as antimicrobial to eradicate infections,
4—Provides wound and skin with excellent nutritional, anti-inflammatory and antioxidants ingredients, and
5—Debridement of dead tissue in wounds and ulcers.

The products are for wound and ulcer treatment, which consist of multi component combination of natural substances. This composition allows batch process production, where it is possible to accomplish mixing and fusion of various natural substances, and cream homogenization, by converting all substances in the dissolvable stage. Honey is used as a semiliquid and can be combined with other ingredients. Hard propolis is converted into dissolvable stage by extraction with ethanol, obtain water extract, or could be used as ground powder. Bee pollen powder is dried and ground into powder. Other ingredients are used as ground powder, or water and alcohol extracts or as an oil form.

One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, wax, beeswax, and the like.

Gels, ointments and creams may be formulated, for example, with an aqueous or oily base with the addition of suitable thickening. Lotions may be formulated with an oily or aqueous base and can contain one or more emulsifying agents, dispersing agents, stabilizing agents, coloring agents, and/or suspending agents and thickening agents.

Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA.

The composition can be applied directly to the wound as a gel, ointment, liquid, cream, or the like as described above. Alternatively, the composition is administered in the form of a wound dressing. Wound dressing may include any one of the different types of substrates and/or backings that are commercially available, including films such as polyurethane films, hydrocolloids such as hydrophilic colloidal particles bound to polyurethane foam, hydrogels, foams such as hydrophilic or hydrophobic, calcium alginates, and cellophane. For example, the composition can be applied to the surface of, or incorporated into a dressing gauze or matrix. Suitable gauze dressings may include, for example, a swab, dry woven or non-woven sponges, bandages and wraps with varying degrees of absorbency. Exemplary fabric compositions may include cotton, rayon or polyester. In certain embodiments, gauzes and non-woven dressings may be available sterile and with or without an adhesive border. In certain embodiments, the dressings also comprise one or more additional pharmaceutically active compounds and/or carrier agents, such as xeroform, oil, saline, zinc salts, petrolatum, and scarlet red. The agent to be used according to the invention may be prepared in all the application forms common for topical medicaments, such as an ointment, shampoo emulsion, stocking, sponge, soap, facial pack, lotion, bath salts, or plaster.

The present composition and/or the components of the composition may be sterilized by any suitable method, including conventional, well-known sterilization techniques, such as autoclaving. Raw, unheated honey can be sterilized, for example, by ozonizing the honey.

The dosage regimen for treating wounds is selected in accordance with a variety of factors including the age, weight, sex, and medical condition of the patient, the severity of the wound, the route of administration, pharmacological considerations such as the activity, efficacy, pharmacokinetic and toxicology profiles of the particular composition used, whether a dressing or drug delivery system is used and whether the composition is administered as part of a drug combination.

The advantageous use of the substance mixture according to the invention consists in preparing an agent for wound and ulcer healing. These properties include anti-inflammatory, antibacterial, and antimycotic effects; wound and ulcer healing activity; supplying vitamins, trace elements, natural glucose, and amino acids. The mixture will provide stronger antioxidants, anti-inflammatory and antimicrobial activities as compared to single ingredient treatments.

Olive oil contains polyphenolic compounds, which has an antibacterial effect to enhance wound healing. The compositions may include deodorized garlic (Allium sativum), such as that described in (U.S. Pat. No. 4,933,201 to Sakai) or pure, powered, pulverized, minced or extract, preferably obtained from the bulb, but not limited to any portion of the plant, preferably derived from the aforementioned member of the genus, but not limited to any species within the Allium genus such as Allium oleraceum, Allium ursinum, Allium ampeloprasum and Allium canadense. The compositions may include pure, solids, decolorized or extract of Turmeric Rhizome (Curcuma Longa), preferably derived from this member of the genus, but not limited to variation in species of the curcuma genus such as Curcuma aromatica and Curcuma amada.

Suggested compositions for treatment of wounds, ulcers, and skin diseases are illustrated in, not limited to, tables 1 and 2.

The invention also relates to a herpes simplex 1 and 2 and zoster healing composition that comprises the topical use of mixture of honey, propolis or CAPE and curcumin (or any two of them) alone or in combination with bee venom or melittin, cloves or clove extract, garlic or AGE, royal jelly or mixed royal jelly, and nigella sativa or thymoquinone in form of gel, ointment or cream. The composition is applied directly on the affected area 3-6 times per day or according to the severity of the lesions. The recipe might include ingredients that help dissolving propolis in water. The healing herein includes curing, alleviating, reversing, palliative and prophylactic treatment of the condition. The composition should be used alone or in combination with drugs used in the management of herpes infection. Multiple compositions can be prepared with mixing any two or more ingredients to achieve the same goals.

This composition allows batch process production, where it is possible mixing and fusion of various natural substances, cream homogenization, by converting all substances in dissolvable stage. Hard propolis is converted into the dissolvable stage by extraction with ethanol, is obtained as a water extract or could be used as ground powder. Bee pollen powder is dried and ground into powder. Honey is used as a semiliquid. Other ingredients are used as ground powder, or water and alcohol extracts or as an oil form. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, wax, beeswax, and the like. Gels, ointments and creams may be formulated, for example, with an aqueous or oily base with the addition of suitable thickening. Lotions may be formulated with an oily or aqueous base and can also contain one or more emulsifying agents, dispersing agents, stabilizing agents, coloring agents, and/or suspending agents thickening agents. Suitable carriers also include creams/ointments conventionally used for topical pharmaceutical preparations, such as alginate, preservatives such as benzyl alcohol, buffers to control pH such as disodium hydrogen phosphate/sodium dihydrogen phosphate, agents to adjust osmolarity such as sodium chloride, and stabilizers such as EDTA. The composition can be applied directly to the wound as a gel, ointment, liquid, cream, or the like as described above.

The main advantages include;
1—Natural products and available everywhere;
2—Safe and almost no side effects;
3—Synergistic effects as herpes healer as well as antimicrobial to eradicate bacterial infections; and
4—Provides the skin with excellent nutritional and anti-inflammatory and antioxidant ingredients.

The invention will be illustrated below with the following example, which is in no way restrictive, illustrated in table 3.

The intravenous administration of mixture of honey, propolis or CAPE and curcumin (or any two of them) alone or mixed with thymoquinone, can be used alone or sequentially or simultaneously with the intravenous antibiotics or cytotoxic agents. When administered simultaneously, the ingredients can be in the same or separate compositions. Other aspects of the invention relate to the use of honey, propolis or CAPE, curcumin, and thymoquinone as suitable forms for parenteral administration.

The intravenous honey, propolis (or CAPE) and curcumin mixture (or any two of them) with or without thymoquinone is for protection of, not limited to, liver, kidney, bone marrow or any other body organs or tissues when used with antibiotics, cytotoxic drugs, and immunosuppressive agents or used in inflammatory diseases, infectious diseases or sepsis.

The amount of the compositions to be administered, the rate of administration and the time of administration will depend on the type of infection to be treated, the antibiotic or cytotoxic agent to be used, and the size, location, progression and/or severity of the infection to be treated. Appropriate dosage for antibiotics or cytotoxic agents, are well known in the art. In some embodiments, honey, propolis (or CAPE) and curcumin mixture (or any two of them) with or without thymoquinone is infused daily or every other day or more than one time daily until antibiotic or cytotoxic agent course is finished. Extension of the honey composition infusion might be considered in such case of recurrent infections or malnutrition of severe infection or sepsis. The formulations can include total honey or honey extract, such as water, alcohol or ethyl acetate extracts.

The methods of the present application are useful for the treatment of various infectious diseases, such as bacterial infection, viral infection, fungal infection or parasitic infection, systemic or local.

The invention will be illustrated below with the following example, which is in no way restrictive.

Honey or honey extract (water, ethanol or ethyl acetate extract): 0.1-2 g/k.b.wt
Curcumin or curcumin extract: 0.1-40 mg/kg
Propolis or propolis extract: 0.1-30 mg/kg.b.wt or CAPE: 0.001-10 mg/kg.b.wt or 0.1-500 micromole/kg.b.wt
Thymoquinone: 0.001-5 mg/kg.b.wt

The compositions may be presented in unit-dose or multi-dose containers. And they may include additional ingredients that are conventional in the art with injectable formulations. The healing herein includes curing, alleviating, reversing, palliative and prophylactic treatment of the condition. Water soluble propolis extract was prepared or using the method described elsewhere (Nikolov N, et al., Bulgarian J Pathology Applied, 1987; 79903/28, 05).

The aims of the composition are:
1—Synergistic effect with antibiotics for treatment of local or systemic infections;
2—For treatment of local or systemic infection
3—For treatment of acute kidney injury and acute liver injury
3—Hydration,
4—Nutrition supplement, and
5—To decrease the side effects of immunosuppressive agents, cytotoxic agents, or antibiotics.

The invention also relates to a peritonitis healing composition, that comprises mixture of honey, propolis or CAPE, curcumin or curcumin extract (or any two of them) alone, or mixed with nigella sativa oil, and/or thymoquinone in the form of a recipe suitable for parenteral administration. It can be used alone, sequentially, or simultaneously with intravenous antibiotics. When administered simultaneously, the ingredients can be in the same or separate compositions. The composition is used for management of acute or chronic bacterial, viral, fungal or parasitic peritonitis. It could be used in liver cirrhosis. The compositions might include any two of the ingredients or all the ingredients together. The compositions might be used alone or in combination with antibiotics.

The amount of the compositions to be administered, the rate of administration and the time of administration will depend on the type of infection to be treated, progression and/or severity of the infection to be treated. The composition is infused daily or every other day or more than one time daily. Extension of the composition infusion or injection might be considered in such case of recurrent infections or malnutrition or severe infection or sepsis.

The main advantages include;
1—Natural products and available everywhere;
2—Safe and almost no side effects;
3—Synergistic effect as an antimicrobial to eradicate infections; and
4—Provides the peritoneum with excellent nutritional and anti-inflammatory and antioxidant ingredients.

A suggested composition for treatment of peritoneal diseases is illustrated in, not limited to, table 4.

The general oral healing composition includes mixture of honey, propolis and curcumin (or any two of them) alone or combined with bee pollen or mixed bee pollen, royal jelly or mixed royal jelly, flaxseed, acacia Arabica, Nigella sativa or thymoquinone or Nigella sativa oil, garlic or aged garlic or AGE, olive oil, cloves or clove extract or clove oil, Flaxseed or flaxseed oil, green tea or green tea extract, walnuts of walnuts oil, ginger or ginger extract, fenugreek seeds or extract, date seeds, why protein, peanuts, for fish oil for treatment of, not limited to, anemia, enhancement of immunity, potentiating the effect of antibiotics and chemotherapy, for management of dyslipidemia, diabetes mellitus, hypertension, and for prevention or amelioration of toxic effects of heavy metals or chemotherapies. Multiple compositions can be prepared with mixing any two or more ingredients including honey, propolis and curcumin mixture to achieve the same goals.

The oral healing composition is a product for oral use to maintain or potentiate the anti-oxidant levels in healthy individuals or in patient during treatment with a chemotherapeutic agent, antibiotics or pain medications such as NSAIDs.

Other aspects of the invention also relate to methods to reduce the toxicity of exposure of antibiotics or cytotoxic agents in a patient, and heavy metals in a patient or healthy individual comprising administering the healing oral formulae to the patient or normal individual with such exposures.

An important aspect relates to a method of reducing the dose of antibiotic or cytotoxic agents administered to a patient for purpose of management of infection, sepsis or malignancies, due to synergistic effects, anti-inflammatory, antioxidant properties and enhancement of immunity, comprising administering the healing oral compositions to the patient.

The compositions may be presented in unit-dose or multi-dose containers. And they may include additional ingredients that are conventional in the art with regard to oral formulations. The healing herein includes curing, alleviating, reversing, palliative and prophylactic treatment of the condition.

The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the mixture according to the invention consists in preparing an agent for increasing immunity, antioxidant capacity and healing cellular power, and also provides an excellent nutritional support in normal individuals and in patients with chronic diseases and infections. The mixture will provide stronger antioxidants, anti-inflammatory and antimicrobial than individual ingredient. These ingredients have anti-cancer properties and immune system stimulating activity.

Chronic disease such as diabetes mellitus, hypertension, cardiovascular diseases, dyslipidemia and immunological diseases are common. There is no cure for any of them, but chronic treatment is necessary to reduce their complications. Nutritional factors, inflammatory mediators and oxidative stress play a major role in the pathogenesis. The oral general healing composition offers the advantage of using ingredients of a natural origin which are readily available and whose association shows excellent high nutritional value, antioxidant and inflammatory property, and antimicrobial activity and anti-tumor activity. The composition of the invention also offers the advantage of being easy to intake orally.

The invention is intended for the management of chronic disease solely or as part of global management, that includes changed lifestyle and modern interventions. The compositions may be presented in unit-dose or multi-dose containers. And they may include additional ingredients that are conventional in the art with oral formulations. The composition of the invention may be in in liquid, semisolid, gel, capsule, or tablet. The invention will be illustrated below with the following examples, which are in no way restrictive.

The main advantages include:
1—Natural products and available everywhere,
2—Safe and almost no side effects,
3—Synergistic effects as a disease healer, and
4—Provides all body system with excellent nutritional, anti-inflammatory and antioxidants ingredients.

A suggested composition for treatment of general medical diseases is illustrated in, not limited to, table 5.

The invention also relates to a renal healing composition, wherein honey, propolis and curcumin (or any two of them) is mixed with flaxseed, acacia Arabica, royal jelly or mixed royal jelly, olive oil, ginger or ginger extract, green tea or green tea extract, bee pollen or mixed bee pollen, garlic or aged garlic or AGE or garlic oil, Nigella sativa or nigella sativa oil or thymoquinone for management of, not limited to, kidney diseases, proteinuria and urinary calculus, and is used as an oral recipe. The dose depends on the stage of the kidney disease, severity of proteinuria, etiology of proteinuria and the comorbidities. The composition presumably alleviates or cures proteinuria and prevents kidney stone formation and facilitates urinary calculus passage. This composition provides a diuretic effect, antioxidants, anti-inflammatory action and analgesic effect. Multiple compositions can be prepared with mixing any two or more ingredients including honey, propolis and curcumin mixture to achieve the same goals.

The compositions may be presented in unit-dose or multi-dose containers. And they may include additional ingredients that are conventional in the art with regard to oral formulations. The healing herein includes curing, alleviating, reversing, palliative and prophylactic treatment of the condition. The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, and the like. The advantageous use of the substance mixture according to the invention consists in preparing the product or composition that will be useful for management of, inter alia, proteinuria due to diabetes, nephritis, nephrotic syndrome, or due to any reason such as heavy metal intoxication, vasculitis, systemic lupus, IgA nephropathy, membranous nephropathy and others causes mentioned in the medical literature.

The mixture will provide stronger antioxidants, anti-inflammatory and antimicrobial than individual ingredients. The treatment goals of this mixture are:
1—healing of proteinuria,
2—healing of kidney injury in acute and chronic kidney diseases, and
3—as part of management of kidney and urinary calculus and crystaluria.

The main advantages include:
1—Natural products and available everywhere,
2—Safe and almost no side effects,
3—Synergistic effects as kidney healer as well as antimicrobial to eradicate infections, and
4—Provides urinary system with excellent nutritional, anti-inflammatory and antioxidants ingredients.

A suggested composition for treatment of kidney and urinary system diseases is illustrated in, not limited to, table 6.

The invention also relates to a H. pylori healing composition comprising mixture of honey, propolis, and curcumin (or any two of them) alone or in combination with acacia Arabica, garlic, Nigella sativa or Nigella sativa oil or thymoquinone, aged garlic or AGE or garlic oil, bee pollen or mixed bee pollen, olive oil, fish oil and cloves or cloves extract in form of oral recipe for management of H. pylori infection. The formulae could be used alone or in combination with antibiotics and a pump inhibitor. The healing herein includes curing, alleviating, reversing, palliative and prophylactic treatment of the condition. The compositions may be presented in unit-dose or multi-dose containers. And they may include additional ingredients that are conventional in the art with oral formulations.

The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the substance mixture according to the invention consists in preparing the product or composition that will be useful to eradicate H. pylori infection, peptic ulcer and dyspepsia. Multiple compositions can be prepared with mixing any two or more ingredients including honey, propolis and curcumin mixture (or any two of them) to achieve the same goals.

The main advantages include:
1—Natural products and available everywhere;
2—Safe and almost no side effects;
3—Synergistic effects to heal H. pylori infection; and
4—Provides the gastrointestinal tract with excellent nutritional, anti-inflammatory and antioxidants ingredients.

A suggested composition for treatment of gastritis and H. pylori infection is illustrated in, not limited to, tables 7.

The invention also relates to a Clostridia difficile colitis healing composition that comprises the composition of mixture of honey, propolis or CAPE and curcumin (or any two of them) alone or with Nigella sativa or Nigella sativa oil or thymoquinone, cloves or cloves extract, garlic or AGE or garlic oil in form of oral recipe. The composition can be used alone or in combination with antibiotics and with or without an intravenous formula presented in the patent. The compositions may be presented in unit-dose or multi-dose containers. And they may include additional ingredients that are conventional in the art with regard to oral formulations. The healing herein includes curing, alleviating, reversing, palliative and prophylactic treatment of the condition. Multiple compositions can be prepared with mixing any two or more ingredients including honey to achieve the same goals. The compositions can be administered by oral route in dosage unit formulations containing conventional nontoxic pharmaceutically acceptable carriers, adjuvants, and vehicles, as desired. One or More pharmaceutically acceptable diluents, excipients or carriers can be added. The composition may comprise substances which assist in its application or storage stability, such as, pharmaceutical adjuvants, stabilizers, buffer substances, preservatives, water, thickening agents, emulsifiers, and the like. The advantageous use of the substance mixture according to the invention consists in preparing the product or composition that will be useful to eradicate clostridia difficile infection.

The main advantages include;
1—Natural products and available everywhere;
2—Safe and almost no side effects;
3—Synergistic effects as clostridia difficile infection healer; and
4—Provides the gastrointestinal system with excellent nutritional and anti-inflammatory and antioxidants ingredients.

A suggested composition for the treatment of colitis and Clostridia difficile colitis is illustrated in, not limited to, table 8.

The invention also relates to a probiotic enhancing composition, wherein the use of mixture of honey, propolis and curcumin (or any two of them) alone or mixed with nigella sativa or thymoquinone or nigella sativa oil, acacia Arabica, royal jelly or mixed royal jelly, garlic or aged garlic or garlic oil, bee pollen or mixed bee pollen, olive oil to stimulate growth of gut bacteria particularly in patients using antibiotic or in patients with colitis. The compositions may be presented in unit-dose or multi-dose containers. And they may include additional ingredients that are conventional in the art with regard to oral formulations. The healing herein includes curing, alleviating, reversing, palliative and prophylactic treatment of the condition. Multiple compositions can be prepared with mixing any two or more than two of the ingredients to achieve the same goals. The compositions can be administered orally in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, beeswax, and the like. The advantageous use of the substance mixture according to the invention consists in preparing the product or composition that will be useful for stimulation of beneficial gut microorganisms in normal individuals and in patients with chronic diseases, on antibiotic management or having colitis or gastrointestinal infections.

The invention is intended to: 1—maintain and enhance the gut microbiota in normal individuals and in patients with antibiotic use or in patients with any diseases related to the partial or total depletion of the gut microbiota, and 2—stimulate gut microbiota growth will help in management of illness.

The main advantages include:
1—Natural products and available everywhere;
2—Safe and almost no side effects;
3—Synergistic effect to stimulate beneficial gut microbial growth; and
4—Provides gastrointestinal tract with excellent nutritional, anti-inflammatory and antioxidant ingredients.

A suggested composition for enhancing gut microbiota (probiotic) is illustrated in, not limited to, table 9.

The invention also relates to an inhalation healing composition, wherein another aspect of the invention relates to use of the mixture of honey, propolis and curcumin (or any two of them) an inhalation composition for management of respiratory diseases. The compositions may be presented in unit-dose or multi-dose containers. And they may include additional ingredients that are conventional in the art with inhalation formulations. The compositions can be administered by inhalation in dosage unit formulations containing conventional nontoxic pharmaceutically acceptable carriers, adjuvants, and vehicles, as desired. One or more pharmaceutically acceptable diluents, excipients or carriers can be added. The composition may comprise substances which assist in its application or storage stability, such as, pharmaceutical adjuvant, stabilizers, buffer substances, preservatives, water, thickening agents, emulsifiers, and the like. The advantageous use of the substance mixture according to the invention consists in preparing the product or composition that will be useful for management of lower and upper respiratory tract infections, sinusitis and obstructive lung diseases. The healing herein includes curing, alleviating, reversing, palliative and prophylactic treatment of the condition. The formulations can include total honey or honey extract, such as water, alcohol or ethyl acetate extracts

The main advantages include;
1—Natural products and available everywhere;
2—Safe and almost no side effects;
3—Synergistic effects as upper and lower respiratory system healer as well as antimicrobial to eradicate infections; and
4—Provides respiratory system with excellent nutritional, anti-inflammatory and antioxidant ingredients.

Honey (comprises from 10-70% w/v honey in suitable solution such as normal saline) is mixed with propolis extract (comprises from 0.1-29% wt/v propolis extract in suitable solution such as normal saline) and with 0.1-1% wt/v curcumin or curcumin extract. Curcumin bioavailability can be increased with incorporation of curcumin with and any substance found to increase curcumin solubility and bioavailability.

The dose could be changed to any other doses found to be more effective with lowest or no side effects.

The composition of the invention also offers the advantage of being easy to use with a nebulizer machine, and the patient inhales the ingredient 1-6 times a day.

The invention is intended for management of lower and upper respiratory tract infections, sinusitis and obstructive lung diseases and can be used alone or with conventional intervention and with or without oral healing formulae. The compositions may be presented in unit-dose or multi-dose containers. And they may include additional ingredients that are conventional in the art with inhalation formulations. The composition of the invention may be in different galenical forms such as in solution form. Various modifications of the composition without departing from the scope may include incorporating new components to optimize efficacy or commercial processing, altering the percent weight of each constituent, introducing isolated chemical compounds derived from each ingredient individually or in combination to produce similar or better therapeutic results, or changing the formulation by subtracting aforementioned ingredients or using various combinations.

Some of the individual ingredients presented in the compositions were tested as inhalers to the respiratory system but the compositions that included mixture of the ingredients such as illustrated in the patent are not published or tested in upper or lower respiratory diseases

Another aspect of the invention is to use mixture of honey, propolis and curcumin (or any two of them including honey) alone as an alternative sweet in normal individuals and in patients with diabetes mellitus. The composition compromises 100% honey or a mixture of 90-99.9% honey and 0.1-5% propolis extract or mixed propolis extract, and 0.5-5% curcumin. The recipe might include ingredients that help dissolving propolis in water. The healing herein includes curing, alleviating, reversing, palliative and prophylactic treatment of the condition. The main advantages include;
1—Natural products and available everywhere;
2—Safe and almost no side effects;
3—Better than sucrose or glucose regarding postprandial blood glucose level; and
4—Provides body with excellent nutritional, anti-inflammatory and antioxidant ingredients.

The invention also comprises the use of mixture of honey, propolis or CAPE and curcumin (or ant two of them) and used by oral administration or parenteral administration for management of parasitic diseases. The composition may include other natural ingredients such as garlic, nigella sativa, thymoquinone, and allicin. The composition is used for management of parasitic diseases such as, not limited to, Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. The formulations can include total honey or honey extract, such as water, alcohol or ethyl acetate extracts.

The main advantages include;
1—Natural products and available everywhere;
2—Safe and almost no side effects;
3—Synergistic effects as a parasitic healer as well as antimicrobial to eradicate infections; and
4—Provides biological systems with excellent nutritional and anti-inflammatory and antioxidant ingredients.

Oral or parenteral compositions are used as part of management of parasitic diseases. The oral composition includes:

Honey: 0.3-2 g/kg.b.wt (or extract of honey at 0.15-1 g/kg).
Propolis or or thr extracts: 0.02-0.14/kg.b.wt
Curcumin or curcumin extract: 0.014-0.40/kg.b.wt
Nigella sativa or its extract: 0.05-0.4/kg.b.wt
Garlic or its extract: 0.014-0.285/kg.b.wt
The extract of ingredients might include water extract, ethanol extract or ethyl acetate extracts or any other suitable extract.

This composition is intended to be part of management of parasitic diseases such as, not limited to, Entamoeba histolytica, Giardia lamella, Blastocystis species, schistosomiasis, malaria, flukes, trypanosome cruzi, hydatid cyst, toxoplasmosis, visceral leishmaniasis, ascaris, nematodes, trematodes, and trichomonas species. Mixed honey can be used alone or mixed with any of the above ingredients.

The parenteral composition includes:

Honey: 0.1-2 g/k.b.wt (Honey extract can be used at 0.15-1 g/kg)
Curcumin or curcumin extract: 0.1-40 mg/kg
Propolis or propolis extract: 0.1-30 mg/kg.b.wt or CAPE: 0.001-10 mg/kg.b.wt or 0.1-500 micromole/kg.b.wt
Thymoquinone: 0.001-5 mg/kg.b.wt
Allicin: 1-5 mg/kg.b.wt
The extract of ingredients might include water extract, ethanol extract or ethyl acetate extracts or any other suitable extract.

The parenteral composition is intended to be part of management of, not limited to, malaria, fluke, liver amoebiasis, trypanosome cruzi, hydatid cyst, toxoplasmosis and visceral or cutaneous leishmaniasis.

### SPECIFIC EXAMPLES OF COMPOSITIONS

The tables below provide suggested compositions of mixed propolis along with other ingredients for treating various conditions. To one skilled in the art, various modifications of the invention, without departing from the scope may include:
1—incorporating new components to optimize efficacy or commercial processing
2—altering the percentage of each constituent
3—introducing isolated chemical compounds derived from each ingredient extract individually or in combination to produce similar or better therapeutic results
4- or changing the formulation by subtracting aforementioned ingredients or using various combinations.

### Wounds and Skin Diseases

The tables below provide suggested compositions of mixture of honey, propolis (or CAPE) and curcumin along with other ingredients for treating wounds, ulcers and skin diseases.

All of the ingredients are natural substances and have various antimicrobial, antioxidant, anti-inflammatory and nutritional properties. Some of the individual ingredients presented in the compositions was tested in wounds and ulcers but the compositions that included mixture of the ingredients such as illustrated in the patent are not published or tested in herpes simplex type 1 and 2 infection or herpes zoster infection

TABLE 1
Mixture of honey, propolis and curcumin based wound healing composition. It includes mixture of honey,
propolis, and curcumin (or any two of them) alone or mixed all the ingredients together, or the mixture
is mixed with any one of the ingredients or mixed with two or more than two of the ingredients. One or
more pharmaceutically acceptable diluents or carriers can be added to adjust the dose such as beeswax.

| | | Concentrations | | | |
|---|---|---|---|---|---|
| | | | Ethanol or water | | |
| | | | extract or ethyl | | |
| Ingredients | | Crude or Powder | acetate extract | oil | Example of formulae |
| Mixture of honey, | Honey | 10-99% | 5-49% | | 50% |
| curcumin and | Curcumin | 5-20% | 5-10% | | 5% |
| propolis | Propolis or | 1-30% | 1-15% | | 10% 0r |
| | mixed | | | | |
| | propolis | | | | |
| | Or CAPE | 0.1-1000 | | | CAPE 50 micromole/100 g |
| | | micromole/100 g | | | |

| | | | | |
|---|---|---|---|---|
| Nigella sativa or | 0.1-15% | 0.1-15% | 0.1-10% | 5% Or |
| Thymoquinone | 0.1-5% | | | 5% thymoquinone |
| Garlic crude or | 5-20% | 1-10% | 0.5-3% | 5% Or |
| Aged Garlic Extract | 1-15% | 1-5% | | 5% aged garlic extract |
| Olive oil | 5-10% | | | 6% |
| Bee pollen powder or mixed bee pollen | 4-10% | 3-6% | | 5% |
| Clove | 1-15% | 0.5-10% | 0.1-10% | 4% |
| Bee venom or | 0.1-15 mg/100 g | 0.1-15 mg/100 g | | 0.1 mg/100 g |
| Melittin | 0.1-5 mg/100 g | 0.1-5 mg/100 g | | or melittin 0.1 mg/100 g |
| Acacia Arabica | 1-20% | 1-10% | | 4% |
| Royal jelly or mixed royal jelly | 1-20% | 5-10% | | 6% |

TABLE 2
Mixture of honey, propolis and curcumin based cutaneous leishmaniasis healing composition.
It includes mixture of honey, propolis, and curcumin (or any two of them) alone or mixed
all the ingredients together, or the mixture is mixed with any one of the ingredients
or mixed with two or more than two of the ingredients. One or more pharmaceutically acceptable
diluents or carriers can be added to adjust the dose such as beeswax.

| | | Concentrations | | | |
|---|---|---|---|---|---|
| | | | Ethanol or water or | | |
| Ingredients | | Crude or Powder | ethyl acetate extract | oil | Example of formulae |
| Mixture of | Honey | 30-70% | 15-35% | | 50% |
| honey, curcumin | Curcumin | 5-20% | 5-10% | | 5% |
| and propolis | Propolis | 1-30% | 1-20% | | 20% Or |
| | or CAPE | 1-1000 | | | CAPE 50 micromole/100 g |
| | | micromole/100 g | | | |

| | | | | |
|---|---|---|---|---|
| Nigella sativa or | 0.1-20% | 0.1-10% | 0.1-10% | 10% Or |
| Thymoquinone | 0.1-10% | | | 10% thymoquinone |
| Garlic crude or | 5-20% | 1-15% | 0.5-3% | 10% Or |
| Aged Garlic Extract | 1-15% | 1-10% | | 10% aged garlic extract |
| Cloves | 1-15% | 0.5-10% | 0.1-10% | 5% |

### Herpes Infection

The patent presents multiple compositions with antiviral, antimicrobial, anti-inflammatory and anti-oxidant activities. The invention will be illustrated below with the following examples, which are in no way restrictive. Some of the individual ingredients presented in the compositions was tested in herpes simplex infections but the compositions that included mixture of the ingredients such as illustrated in the patent are not published or tested in herpes simplex type 1 and 2 infection or herpes zoster infection.

TABLE 3
Mixture of honey, propolis and curcumin based herpes healing composition. It includes mixture of honey,
propolis, and curcumin (or any two of them) alone or mixed all the ingredients together, or the mixture
is mixed with any one of the ingredients or mixed with two or more than two of the ingredients. One or
more pharmaceutically acceptable diluents or carriers can be added to adjust the dose such as beeswax.

| | | Concentrations | | | |
|---|---|---|---|---|---|
| | | | Ethanol or water or | | |
| Ingredients | | Crude or Powder | ethyl acetate extract | oil | Example of formulae |
| Mixture of | Honey | 30-99% | 15-49% | | 60% |
| honey, | Curcumin | 5-20% | 5-10% | | 10% |
| curcumin | Propolis or | 1-30% | 1-15% | | 10% Or |
| and propolis | CAPE | 1-100 | | | CAPE 50 micromole/100 g |
| | | micromole/100 g | | | |

| | | | | |
|---|---|---|---|---|
| Nigella sativa | 0.1-5% | 0.1-5% | | 5% |
| Or Thymoquinone | | | | |
| Cloves | 1-15% | 0.5-10% | 0.1-10% | 5% |
| Bee venom or | 0.1-15 mg/100 g | 0.1-15 mg/100 g | | 0.2 mg/100 g or melittin |
| Melittin | 0.1-5 mg/100 g | 0.1-5 mg/100 g | | 0.1 mg/100 g |
| Royal jelly or mixed royal jelly | 1-20% | 5-10% | | 6% |
| Garlic or AGE | 5-20% or AGE 1-15% | 1-10% | 0.5-3% | 5% |

### Intraperitoneal Infection

The patent presents new composition with natural ingredients that provides excellent antimicrobial, antioxidants and anti-inflammatory intervention. A peritonitis healing composition can be created using the table below:

TABLE 4
Mixture of honey, propolis and curcumin based peritonitis
healing composition. It includes mixture of honey, propolis,
and curcumin (or any two of them) alone or mixed all the
ingredients together, or the mixture is mixed with any one
of the ingredients or mixed with two or more than two of
the ingredients. One or more pharmaceutically acceptable
diluents or carriers can be added to adjust the dose.

| | | Doses | | Example of | |
|---|---|---|---|---|---|
| Ingredients | | (per kg/day) | | the formulae | |
| Mixture of honey, | Honey | 1-2 | g | 70 | g |
| curcumin and | Or honey extract in | 0.5-1 | g | | |
| propolis | water, ethanol or | | | | |
| | ethyl acetate | | | | |
| | Curcumin extract | 0.001-0.1 | g | 1 | g |
| | CAPE or | 0.001-0.03 | g | 0.5 | g |
| | Propolis extract | 0.005-0.1 | g | 1 | g |

| | | | | |
|---|---|---|---|---|
| Thymoquinone | 0.002-0.02 | g | 0.5 | g |
| Nigella sativa extract Or | 0.005-0.1 | g | 1 | g |
| Nigella sativa oil | 0.0142-0.5 | ml | 5 | ml |

Daily dose of the formulae 0.5-3 gram/kg · b · wt.

### Chronic Diseases and General Oral Composition

Some of the individual ingredients presented in the compositions were tested in the chronic diseases but the compositions that included mixture of the ingredients such as illustrated in the patent are not published or tested in the chronic or acute diseases intended to be treated by the compositions. Furthermore, mixture of honey, propolis and curcumin was never tested in management of acute or chronic disease.

TABLE 5
Mixture of honey, propolis and curcumin based general oral healing formulae. It
includes mixture of honey, propolis, and curcumin (or any two of them) alone or
mixed all the ingredients together, or the mixture is mixed with any one of the
ingredients or mixed with two or more than two of the ingredients. One or more pharmaceutically
acceptable diluents or carriers can be added to adjust the dose.

| | | Doses per 70 kg · b · wt/day | | |
|---|---|---|---|---|
| | | | | Example of the |
| Ingredients | | Gram/kg · b · wt/day | Gram/day | formulae gram |
| Mixture of honey, | honey or | 0.1-2 | 7-140 | 50 |
| curcumin and | Honey extract | 0.5-1 | 3.5-70 | 25 |
| propolis | with water, | | | |
| | ethanol or ethyl | | | |
| | acetate | | | |
| | Curcumin or | 0.007-0.42 | 0.5-20 | 10 |
| | curcumin extract | | | |
| | Propolis Or | 0.011-0.285 | 0.8-20 | 5 |
| | propolis extract Or | 0.005-0.21 | 0.4-15 | 5 |
| | CAPE | 0.001-003 | 0.07-0.21 | 0.1 |

| | | | |
|---|---|---|---|
| Royal jelly or mixed royal jelly | 0.014-0.85 | 1-6 | 5 |
| Nigella sativa | 0.007-0.28 | 0.5-20 | 5 |
| Or Thymoquinone | 0.001-0.4 | 0.07-2.8 | 1 |
| Or Nigella sativa oil | 0.014-0.14 | 0.1-10 | 3 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Propolis | 0.011-0.285 | 0.8-20 | 5 |
| Or propolis extract | 0.005-0.21 | 0.4-15 | 5 |
| Or CAPE | 0.001-003 | 0.07-0.21 | 0.1 |
| Bee pollen or mixed bee pollen | 0.142-0.57 | 10-40 | 15 |
| Olive oil | 0.142-0.57 | 10-40 | 20 |
| Cloves or cloves extract | 0.007-0.14 | 0.5-10 | 3 or |
| Or clove oil | 0.014-0.14 | 1-10 | clove oil 2 ml |
| Flaxseed | 0.142-0.57 | 10-40 | 10 |
| Or flaxseed oil | 0.014-0.085 | 1-6 | 1 |
| Green tea or green tea extract | 0.007-0.071 | 0.5-5 | 1 |
| Fish oil | 0.014-0.042 | 1-3 | 2 |
| Walnuts | 0.011-0.71 | 20-50 Or | 20 Or |
| Or walnuts oil | 0.014-0.071 | oil 1-5 | oil 3 |
| Ginger powder | 0.014-0.285 | 1-20 | 5 Or |
| Or Ginger water extract | 0.014-0.285 | 1-20 | water extract 3 |
| Or Ginger ethanol extract | 0.0014-0.15 | 0.1-10 | Or ethanol extract 2 |
| Fenugreek seeds powder | 0.028-0.214 | 2-15 | 5 Or |
| Or extract | 0.004-0.028 | 0.3-2 | extract 1 |

Daily dose of the formulae 0.5-3 gram/kg · b · wt. Other ingredients of the mixtures might include date seed powder (5-20%), why protein (5-20%), peanuts or any other type of nuts (5-20).

### Kidney and Urinary System Diseases

The renal healing composition according to the invention offers the advantage of using ingredients of a natural origin which are readily available and whose association shows excellent high nutritional value, renal protection property, potent antioxidant activity, anti-inflammatory action, diuretic activity, urinary nitric oxide stimulation, and antimicrobial activity. The composition can also ameliorate proteinuria. The composition of the invention also offers the advantage of being easy to use as an oral recipe. The invention is intended for the treatment of proteinuria, urinary crystaluria, urinary calculus, and acute and chronic kidney failure due to, but not limited to, diabetic nephropathy, autoimmune disorders, cytotoxic or immunosuppressive agents, or infectious causes. The compositions may be presented in unit-dose or multi-dose containers. And they may include additional ingredients that are conventional in the art with oral formulations. The composition of the invention may be in different galenical forms gel, liquid, or semiliquid.
The invention will be illustrated below with the following examples, which are in no way restrictive.

TABLE 6
Mixture of honey, propolis and curcumin based kidney composition. It includes
mixture of honey, propolis, and curcumin (or any two of the) alone or mixed all
the ingredients together, or the mixture is mixed with any one of the ingredients
or mixed with two or more than two of the ingredients. One or more pharmaceutically
acceptable diluents or carriers can be added to adjust the dose.

| | | Doses | | |
|---|---|---|---|---|
| | | | | Example of the |
| Ingredients | | Gram/kg · b · wt | Gram/day | formulae |
| Mixture of honey, | Honey or | 0.1-2 | 7-140 | 50 |
| propolis and | Honey extract with | .05-1 | 3.5-70 | 25 |
| curcumin | water, alcohol or | | | |
| | ethyl acetate | | | |
| | Curcumin or | 0.014-0.42 | 1-30 | 20 |
| | curcumin extract | | | |
| | Propolis Or | 0.011-0.141 | 0.8-10 | 5 |
| | propolis extract Or | 0.005-0.141 | 0.4-10 | 5 |
| | CAPE | 0.001-003 | 0.07-0.21 | 0.1 |

| | | | |
|---|---|---|---|
| Royal jelly or mixed royal jelly | 0.014-0.085 | 1-6 | 5 |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Acacia Arabica | 0.07-0.42 | 1-30 | 10 |
| Garlic | 0.014-0.285 | 1-20 | 5 |
| Or aged garlic extract | 0.014-0.14 | 1-10 | 5 |
| Or garlic oil | 0.0014-0.071 | 0.1-5 | 0.5 |
| Bee pollen or mixed bee pollen | 0.142-0.57 | 10-40 | 20 |
| Olive oil | 0.142-0.57 | 100-40 | 30 |
| Flaxseed | 0.142-0.57 | 10-40 | 10 |
| Or flaxseed oil | 0.014-0.085 | 1-6 | 1 |
| Ginger powder | 0.014-0.285 | 1-20 | 5 Or |
| Or ginger water extract | 0.014-0.285 | 1-20 | water extract 3 |
| Or ginger ethanol extract | 0.014-0.15 | 0.1-10 | Or ethanol extract 2 |
| Green tea or green tea extract | 0.0071-0.071 | 0.5-5 | 1 |

Daily dose of the composition 0.5-3 gram/kg · b · wt.

Some of the individual ingredients presented in the compositions were tested in kidney diseases but the compositions that included mixture of the ingredients such as illustrated in the patent are not published or tested in the acute or chronic kidney diseases.

H. pylori Infection

It was estimated that H. pylori infects more than half of the human population worldwide. It causes chronic active gastritis, peptic ulcer disease and gastric cancer. It is involved in the development of peptic ulcer disease in 10%-20%, and gastric lymphoma in 1%-2% of the infected individuals.

The patent presents various compositions with antimicrobial, anti-inflammatory and antioxidant activity for treatment of H. pylori-induced diseases and for eradication of the infection. The invention will be illustrated below with the following examples, which are in no way restrictive.

TABLE 7
Mixture of honey, propolis and curcumin based H. pylori composition. It includes
mixture of honey, propolis, and curcumin (or any two of them) alone or mixed all
the ingredients together, or the mixture is mixed with any one of the ingredients
or mixed with two or more than two of the ingredients. One or more pharmaceutically
acceptable diluents or carriers can be added to adjust the dose.

| | | Doses per 70 kg · b · wt/day | | |
|---|---|---|---|---|
| | | | | Example of the |
| Ingredients | | Gram/kg · b · wt/day | Gram/day | formulae/gram |
| Mixture of honey, | Honey or | 0.1-2 | 7-140 | 50 |
| curcumin and | Honey extract with | .05-1 | | |
| propolis | water, ethanol or | | | |
| | ethyl acetate | | | |
| | Curcumin or | 0.014-0.42 | 1-20 | 10 |
| | curcumin extract | | | |
| | Propolis Or | 0.011-0.285 | 0.8-20 | 5 |
| | propolis extract Or | 0.005-0.21 | 0.4-15 | 5 |
| | CAPE | 0.001-003 | 0.07-0.21 | 0.1 |

| | | | |
|---|---|---|---|
| Nigella sativa | 0.014-0.28 | 1-20 | 5 |
| Or Thymoquinone | 0.001-0.4 | 0.07-2.8 | 1 |
| Or Nigella sativa oil | 0.014-0.14 | 0.1-10 | 3 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Bee pollen or mixed bee pollen | 0.142-0.57 | 10-40 | 15 |
| Olive oil | 0.142-0.57 | 100-40 | 20 |
| Cloves or cloves extract | 0.007-0.14 | 0.5-10 | 1 Or |
| Or clove oil | 0.014-0.14 ml | 1-10 ml | clove oil 2 ml |
| Fish oil | 0.014-0.042 | 1-3 | 2 |

Daily dose of the composition 0.5-3 gram/kg · b · wt.

Some of the individual ingredients presented in the compositions were tested in H. pylori infection but the compositions that included mixture of the ingredients such as illustrated in the patent are not published or tested in the H. pylori infection or dyspepsia.
Clostridium difficile Colitis

TABLE 8
Mixture of honey, propolis and curcumin based Clostridium difficile colitis composition.
It includes mixture of honey, propolis, and curcumin (or any two of them) alone or
mixed all the ingredients together, or the mixture is mixed with any one of the ingredients
or mixed with two or more than two of the ingredients. One or more pharmaceutically
acceptable diluents or carriers can be added to adjust the dose

| | | Doses per 70 kg · b · wt/day | | |
|---|---|---|---|---|
| | | | | Example of the |
| Ingredients | | Gram/kg · b · wt/day | Gram/day | formulae gram |
| Mixture of honey, | Honey or | 0.1-2 | 7-140 | 50 |
| curcumin and | Honey extract with | .05-1 | 3.5-70 | 25 |
| propolis | water, alcohol or | | | |
| | ethyl acetate | | | |
| | Curcumin or | 0.014-0.42 | 1-30 | 20 |
| | curcumin extract | | | |
| | Propolis Or | 0.011-0.285 | 0.8-20 | 5 |
| | propolis extract Or | 0.005-0.21 | 0.4-15 | 5 |
| | CAPE | 0.001-003 | 0.07-0.21 | 0.1 |

| | | | |
|---|---|---|---|
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Cloves or cloves extract | 0.007-0.14 | 0.5-10 | 3 |

Daily dose of the formulae 0.5-3 gram/kg · b · wt.

### Probiotics—Gut Bacteria

The gut microbiota are important for vitamin biosynthesis, bile acid degradation, carbohydrate digestion, contribution of nutrients, and regulation of intestinal hormones. Gut microbiota play an important role in human diseases, such as obesity, fatty liver disease, insulin resistance/diabetes, and cardiovascular events.

The invention will be illustrated below with the following examples, which are in no way restrictive.

TABLE 9
Mixture of honey, propolis and curcumin based probiotic enhancing composition It
includes mixture of honey, propolis, and curcumin (or any two of them) alone or
mixed all the ingredients together, or the mixture is mixed with any one of the
ingredients or mixed with two or more than two of the ingredients. One or more
pharmaceutically acceptable diluents or carriers can be added to adjust the dose.

| | | Doses per 70 kg · b · wt/day | | |
|---|---|---|---|---|
| | | | | Example of the |
| Ingredients | | Gram/kg · b · wt/day | Gram/day | formulae gram |
| Mixture of honey, | Honey Or | 0.1-2 | 21-140 | 50 |
| curcumin and | Honey extract with | 0.05-1 | | |
| propolis | water, alcohol or | | | |
| | ethyl acetate | | | |
| | Curcumin or | 0.014-0.42 | 1-30 | 20 |
| | curcumin extract | | | |
| | Propolis Or | 0.011-0.285 | 0.8-20 | 5 |
| | propolis extract Or | 0.005-0.21 | 0.4-15 | 5 |
| | CAPE | 0.001-003 | 0.07-0.21 | 0.1 |

| | | | |
|---|---|---|---|
| Royal jelly or mixed royal jelly | 0.014-0.85 | 1-6 | 5 |
| Nigella sativa | 0.014-0.28 | 1-20 | 10 |
| Or Thymoquinone | 0.001-0.4 | 0.07-2.8 | 1 |
| Or Nigella sativa oil | 0.014-0.14 | 0.1-10 | 5 |
| Acacia Arabica | 0.07-0.42 | 1-30 | 10 |
| Garlic | 0.014-0.285 | 1-20 | 5 |
| Or aged garlic extract | 0.014-0.85 | 1-6 | 5 |
| Or garlic oil | 0.0014-0.071 | 0.1-5 | 0.5 |
| Bee pollen or mixed bee pollen | 0.142-0.57 | 10-40 | 15 |
| Olive oil | 0.142-0.57 | 100-40 | 20 |

Daily dose of the formulae 0.5-3 gram/kg · b · wt.

Some of the individual ingredients presented in the compositions were tested in growth of gut bacteria but the compositions that included mixture of the ingredients such as illustrated in the patent are not published or tested to stimulate growth of gut bacteria.

Accordingly, while only a few embodiments of the present invention have been shown and described, it is obvious that many changes and modifications may be made thereunto without departing from the spirit and scope of the invention.

What is claimed is:

1. A composition for treatment of a medical condition, comprising a mixture of at least two of the following components: honey, curcumin and propolis (or CAPE).

2. The composition according to claim 1, further comprising at least one of the following additional ingredients: bee venom or melittin, royal jelly or mixed royal jelly, bee pollen or mixed bee pollen, acacia Arabica, nigella sativa or thymoquinone, clove or clove oil, olive oil, garlic, flaxseed or flaxseed oil, fish oil, green tea, fenugreek seeds, walnuts, date seeds, whey protein, peanuts, other nuts, and ginger.

3. The composition according to claim 1, wherein the the mixture contains any two of the following components: honey in an amount of 10-99 wt %, curcumin in an amount of 5-20 wt % and propolis in amount of 1-30 wt % or CAPE in amount of 0.1-1000 micromole/100 g.

4. The composition according to claim 1, wherein the composition is formulated for topical application to the skin.

5. The composition according to claim 1, wherein the composition is configured for parenteral administration.

6. The composition according to claim 1, wherein the composition is configured for inhalation.

7. The composition according to claim 1, wherein the composition is configured for oral administration.

8. The composition according to claim 4, wherein the composition is in the form of a gel, ointment, spray, suppository, liquid or cream.

9. The composition according to claim 4, wherein the composition is disposed on a surface of a dressing for wound care.

10. The composition according to claim 2, wherein the composition is a wound healing composition configured for topical application and contains honey in an amount of 10-99 wt % or extract of honey in water, ethanol or ethyl acetate in an amount of 5-45%, curucmin in amount of 5-20 wt % or curcumin extract in water, ethanol or ethylacetate in amount of 5-10 wt %, and propolis in amount of 1-30 wt % or propolis extract in amount of 1-15%, or CAPE in amount of 1-1000 micromole/100 g, and at least one of the following ingredients in proportions by weight:
Nigella sativa or extracts of nigella sativa: 0.1-15%
Or thymoquinone: 0.1-5%;
Garlic crude: 5-20% or as an extract: 1-10%;
Olive oil: 5-10%;
Bee pollen powder (single or mixed): 4-10%
or extract of bee pollen: 3-6%;
Cloves: 1-15% or extract of cloves: 0.5-10%;
Bee venom or its extract: 0.1-15 mg/100 g or
Melittin: 0.5-5 mg/100 g:
Acacia Arabica: 1-20%; or its extract 1-10%
Royal jelly (single or mixed): 1-20% or extract: 5-10%.

11. The composition according to claim 2, wherein the composition is a kidney healing composition configured for oral administration for treatment of kidney diseases and contains honey in an amount of 0.1-2 gram/Kg.b.wt or extract of honey in water, ethanol or ethyl acetate in an amount of 0.04-1 gram/kg.b.wt, curucmin or curcumin extract in amount of 0.014-0.42 g/kg.b.wt, and propolis in amount of 1-30 wt % or propolis extract in amount of 0.011-0.141 gram/kg.b.wt or its extract 0.005-0.141 gram/kg.b.wt or CAPE in amount of 0.001-0.003 gram/kg.b.wt, and at least one of the following ingredients in proportions by weight:
Royal jelly or mixed royal jelly 0.014-0.085;
Nigella sativa 0.014-0.28, or Thymoquinone 0.001-0.4, or Nigella sativa oil 0.014-0.14;
Acacia Arabica 0.07-0.42;
Garlic 0.014-0.285; or aged garlic extract 0.014-0.14, or garlic oil 0.0014-0.071;
Bee pollen or mixed bee pollen 0.142-0.57;
Olive oil 0.142-0.57;
Flaxseed 0.142-0.57 or flaxseed oil 0.014-0.085;
Ginger powder 0.014-0.285 or ginger water extract 0.014-0.285 or ginger ethanol extract 0.014-0.15;
Green tea or green tea extract 0.0071-0.071.

12. The composition according to claim 2, wherein the composition is configured for intravenous administration and contains the following ingredients in the following amounts:
Honey or an extract of honey in water, ethanol or ethyl acetate: 0.1-2 g/k.b.wt.
Curcumin or curcumin extract: 0.1-40 mg/kg.b.wt.
Propolis or extract of propolis: 0.1-30 mg/kg.b.wt; or CAPE: 0.001-10 mg/kg.b.wt or 0.1-500 micromole/kg.b.wt.
Thymoquinone: 0.001-5 mg/kg.b.wt.

13. The composition according to claim 2, wherein the composition is configured for intraperitoneal administration and contains the following ingredients in the following amounts per kg.b.wt.
Honey: 1-2 g or honey extract in water, ethanol or ethyl acetate 0.5-1 g.
Curcumin extract: 0.001-0.1 g.
Propolis extract 0.005-0.1 g or CAPE: 0.001-0.03 g.
Nigella sativa extract: 0.005-0.1 g or Nigella sativa oil 0.0142-0.5 ml, or Thymoquinone: 0.002-0.02 g.

14. The composition according to claim 2, wherein the composition is a herpes healing composition configured for topical application and contains honey in an amount of 10-99 wt % or extract of honey in water, ethanol or ethyl acetate in an amount of 5-45%, curcumin in amount of 5-20 wt % or curcumin extract in water, ethanol or ethylacetate in amount of 5-10 wt %, and propolis in amount of 1-30 wt % or propolis extract in amount of 1-15%, or CAPE in amount of 1-1000 micromole/100 g, and at least one of the following ingredients in proportions by weight:
Nigella sativa or extracts of nigella sativa: 0.1-15%
Or thymoquinone: 0.1-5%.
Garlic crude: 5-20% or AGE (1-15%) or as an extract: 1-10%.
Cloves: 1-15% or extract of cloves: 0.5-10%, or oil 0.1-10%.
Bee venom or extract: 0.1-15 mg/100 g or Melittin 0.1-5 mg/100 g or extract: 0.5-5 mg/100 g.
Royal jelly (single or mixed): 1-20% or extract: 5-10%.

15. A method for treating a medical condition, comprising administering to a subject in need thereof a composition comprising a mixture of honey or honey extract, curcumin or curcumin extract, and propolis or propolis extract or CAPE.

16. The method according to claim 15, wherein the composition is administered by one of the following methods: orally, inhalation, parenterally, and topically.

17. The method according to claim 15, wherein the composition additionally contains at least one of the following ingredients: bee venom or melittin, royal jelly or mixed royal jelly, bee pollen or mixed bee pollen, acacia Arabica, nigella sativa or thymoquinone, cloves or clove oil, olive oil, fish oil, garlic, flaxseed or flaxseed oil, green tea, fenugreek seeds, walnuts, date seed, whey protein, peanuts, other nuts, and ginger.

18. The method according to claim 17, wherein the medical condition is one selected from the group consisting of wounds, burns, ulcers, skin infection, contact dermatitis, eczema, psoriasis, wrinkled skin, allergic reactions, herpes simplex, herpes zoster, cutaneous leishmaniasis, C. diff colitis, peritonitis, proteinuria, urinary calculus, anemia, for potentiating the effects of antibiotics and chemotherapy, dyslipidemia, diabetes mellitus, cardiovascular diseases, H. pylori gastritis or ulcers, dyspepsia, acute and chronic kidney diseases, hypertension, amelioration of toxic effects of heavy metals or chemotherapies or radio therapy, infectious diseases, parasitic diseases, sepsis, dehydration, malnutrition, sinusitis, lung obstructive diseases and malignancies, and as a sweetener.
